(19) United States
(12) Patent Application Publication
(10) Pub. No.: US 2025/0248698 A1
(43) Pub. Date: Aug. 7, 2025

(54) DILATION AND BIOPSY APPARATUS

(71) Applicant: Marisel Chibas, El Centro, CA (US)

(72) Inventor: Marisel Chibas, El Centro, CA (US)

(21) Appl. No.: 18/434,453

(22) Filed: Feb. 6, 2024

(51) Int. Cl. A61B 10/06; A61B 10/02; A61B 17/00; A61B 17/02

(52) U.S. Cl. CPC A61B 10/06; A61B 10/0266; A61B 10/0291; A61B 17/0218; A61B 2010/0208; A61B 2017/00367

(57) ABSTRACT

A dilation and biopsy apparatus for dilating a lumen to facilitate inserting forceps jaws into the lumen includes a forceps with a shaft and a pair of jaws. The jaws are pivotally coupled to a front end of the shaft to alternately open and close. A dilator is mounted to the shaft of the forceps. The dilator comprises a rod and an insertion member. The rod is positioned in the shaft and is linearly movable alternately forwardly and rearwardly with respect to the shaft. The insertion member is coupled to a forward end of the rod and is movable alternately into and out of the shaft through the front end of the shaft and between the pair of jaws.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to dilator apparatuses and forceps and more particularly pertains to a new dilator apparatus for dilating a lumen to facilitate inserting forceps jaws into the lumen.

### (2) Description of Related Art Including Information Disclosed Under 37 CFR 1.97 and 1.98

Collection of biopsy tissue from a cervix is an important step in detecting precancerous and cancerous changes to the cervix. To collect such tissue, it may be desirable to obtain tissue from the endocervical canal of a patient. This is because almost all manifestations of cervical carcinogenesis occur in an area of the cervix called the Transformation Zone, which is located within the endocervical canal in some patients. In order to obtain tissue from the endocervical canal, the orifice leading to the endocervical canal from the vaginal canal, called the cervical os, requires dilation to insert forceps or other biopsy tools into the endocervical canal.

Apparatuses of the prior art which dilate the cervical os include sets of increasingly wide rods which are successively inserted into the endocervical canal through the cervical os during biopsy procedures. Such procedures can be time consuming due to the several rods which must be inserted and removed. An apparatus which uses a single dilation tool to gradually dilate the cervical os and which is incorporated into a forceps would therefore be advantageous to speed cervical biopsy procedures and minimize the time during which patients must endure the discomfort which follows from such procedures.

### BRIEF SUMMARY OF THE INVENTION

An embodiment of the disclosure meets the needs presented above by generally comprising a forceps with a shaft and a pair of jaws. The jaws are pivotally coupled to a front end of the shaft to alternately open and close. A dilator is mounted to the shaft of the forceps. The dilator comprises a rod and an insertion member. The rod is positioned in the shaft and is linearly movable alternately forwardly and rearwardly with respect to the shaft. The insertion member is coupled to a forward end of the rod and is movable alternately into and out of the shaft through the front end of the shaft and between the pair of jaws.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a dilation and biopsy apparatus according to an embodiment of the disclosure.

FIG. 2 is a detail perspective view of an insertion member of an embodiment of the disclosure.

FIG. 3 is a detail perspective view of an insertion member of an embodiment of the disclosure.

FIG. 4 is a perspective view of an embodiment of the disclosure with a pair of jaws of a forceps closed.

FIG. 5 is a perspective view of an embodiment of the disclosure with a view of jaws of a forceps open.

FIG. 6 is a cross-sectional view of an embodiment of the disclosure taken from Arrows 6-6 in FIG. 4.

FIG. 7 is a detail view of an embodiment of the disclosure taken from FIG. 6.

FIG. 8 is an in-use view of an embodiment of the disclosure dilating a cervical OS.

FIG. 9 is an in-use view of an embodiment of the disclosure taking a biopsy tissue of a cervix from an endocervical canal.

### DETAILED DESCRIPTION OF THE INVENTION

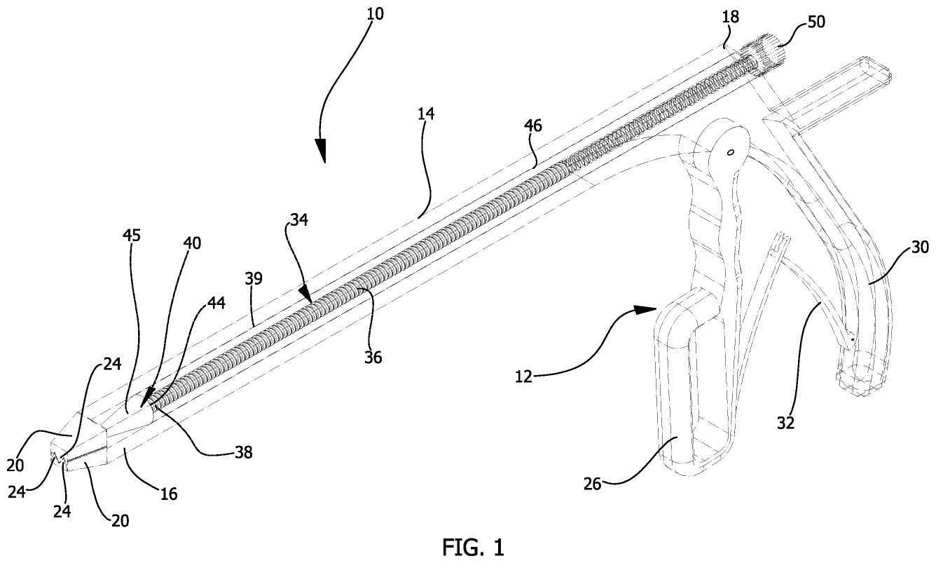

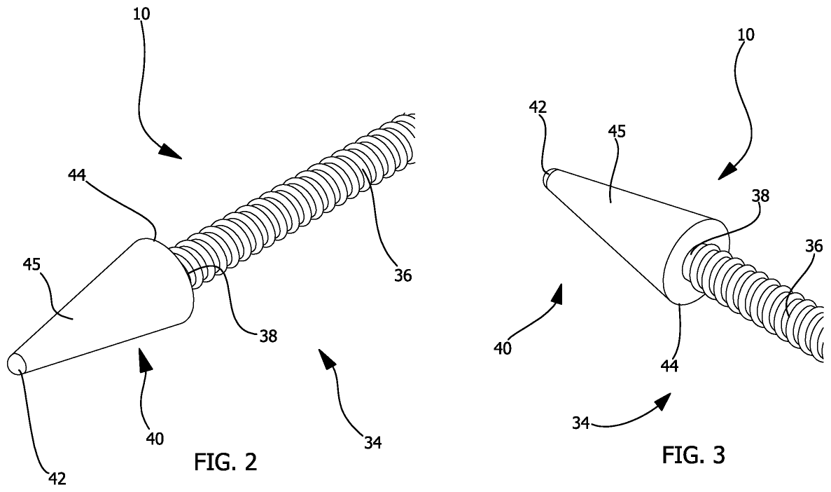

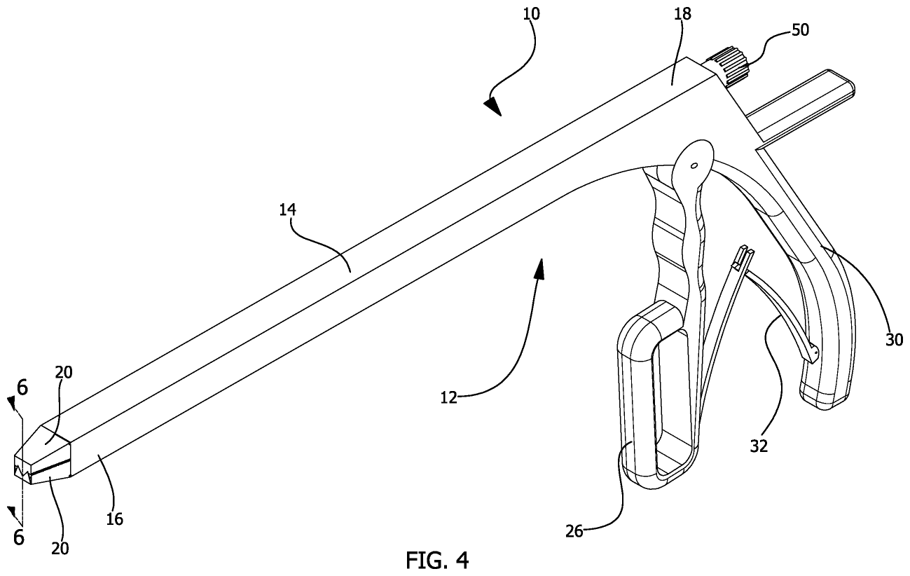

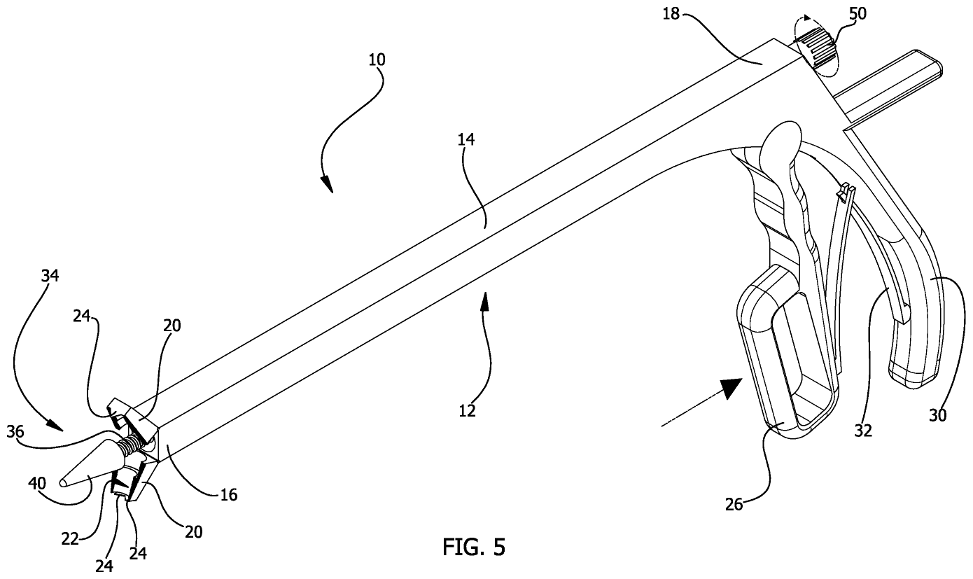

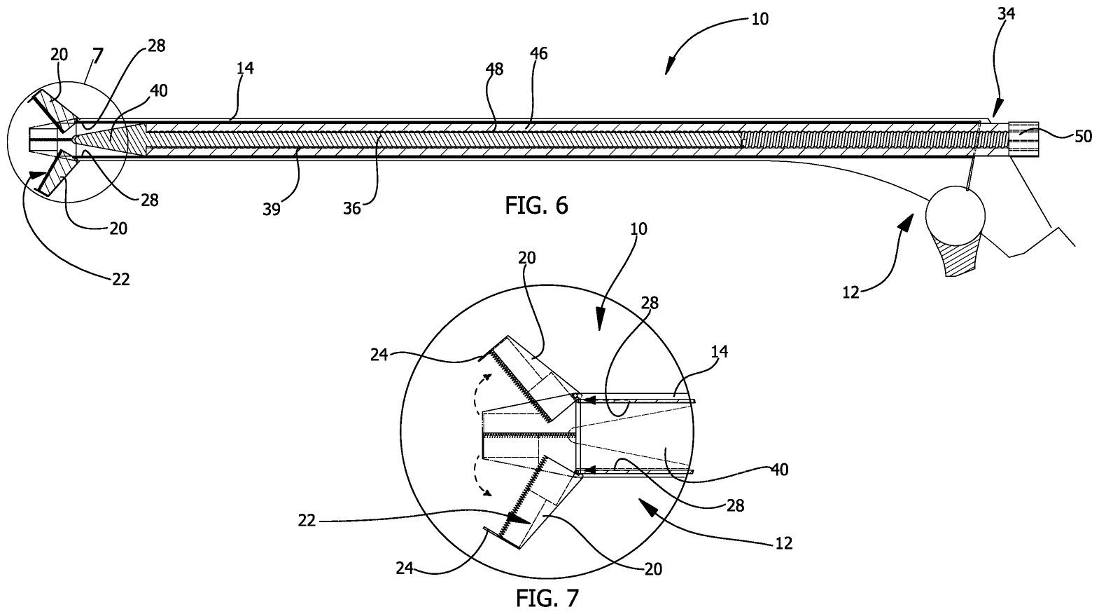

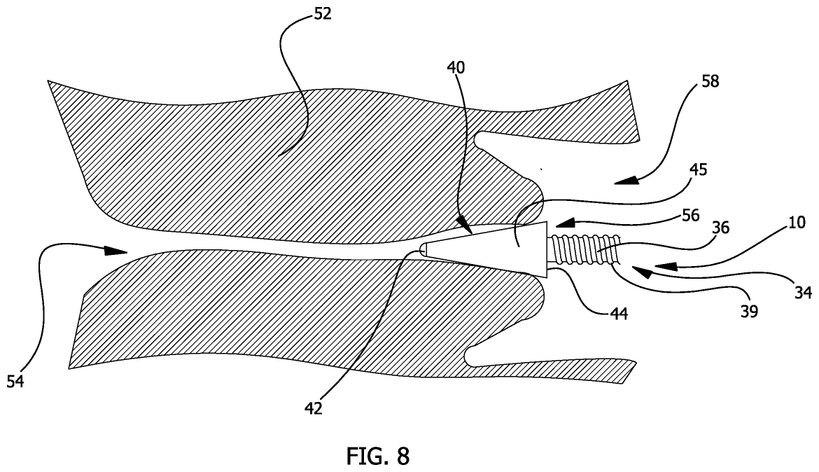

With reference now to the drawings, and in particular to FIGS. 1 through 9 thereof, a new dilator apparatus embodying the principles and concepts of an embodiment of the disclosure and generally designated by the reference numeral 10 will be described.

As best illustrated in FIGS. 1 through 9, the dilation and biopsy apparatus 10 generally comprises a forceps 12 comprising a shaft 14 and a pair of jaws 20 pivotally coupled to a front end 16 of the shaft 14. The jaws 20 are pivotable to alternately open and close and are shaped to define a space 22 therebetween that has a size such that the pair of jaws 20 are configured to hold a biopsy tissue 60 in the space 22. The pair of jaws 20 has interlocking teeth 24 positioned distally from the shaft 14. The interlocking teeth 24 have cutting edges such that the interlocking teeth 24 are configured for severing the biopsy tissue 60 from a target lumen when the pair of jaws 20 close onto the biopsy tissue 60.

The forceps 12 further comprises a trigger 26, a pair of actuator arms 28, a handle 30, and a bias member 32. The trigger 26 is coupled to a rear end 18 of the shaft 14 and is operatively coupled to the pair of jaws 20 to alternately open and close the jaws 20 via the pair of actuator arms 28. The actuator arms 28 are slidably mounted in the shaft 14, and each actuator arm 28 is coupled to the trigger 26 and an associated jaw 20 of the pair of jaws 20. The trigger 26 is pivotable to cause the pair of actuator arms 28 to move linearly through the shaft 14 and urge the pair of jaws 20 to alternately open and close. The handle 30 is coupled to the rear end 18 of the shaft 14 and the trigger 26 is pivotable alternately toward and away from the handle 30 to operate the pair of jaws 20. The bias member 32 is coupled to the trigger 26 and the handle 30 to bias the trigger 26 away from the handle 30, thereby causing the pair of jaws 20 to close.

A dilator 34 is mounted to the shaft 14 of the forceps 12 and comprises a rod 36 and an insertion member 40. The rod 36 is positioned in the shaft 14 and is linearly movable alternately forwardly and rearwardly with respect to the shaft 14. The insertion member 40 is coupled to a forward end 38 of the rod 36. The insertion member 40 has a conical shape, wherein a tip 42 of the insertion member 40 points forwardly with respect to the shaft 14. The insertion member 40 is movable alternately into and out of the shaft 14 through the front end 16 of the shaft 14 and between the pair of jaws 20. A base 44 of the insertion member 40 has a width of at most 0.30 inches, and the insertion member 40 has a length from the tip 42 to the base 44 of at most 0.60 inches.

A rod actuator 46 is positioned in the shaft 14 and is movable to move the rod 36. The rod actuator 46 has internal threads 48 which engage external threads 39 of the rod 36, whereby the rod actuator 46 is rotatable to cause the rod 36 to move linearly with respect to the shaft 14. Keyways or similar tracks may be employed to guide the rod 36 to move linearly when the rod actuator 46 is rotated. A knob 50 is coupled to the rod actuator 46 and is positioned rearwardly of the shaft 14. The knob 50 may be rotated to cause the rod actuator 46 to rotate.

In use, the dilator 34 is inserted into the target lumen to dilate an open end of the target lumen. The dilator 34 may then be retracted into the forceps 12, and the jaws 20 of the forceps 12 are inserted into the target lumen through the widened open end to retrieve the biopsy tissue 60. In particular, the dilation and biopsy apparatus 10 may be used to sample tissue from an endocervical canal 54 as described more fully below.

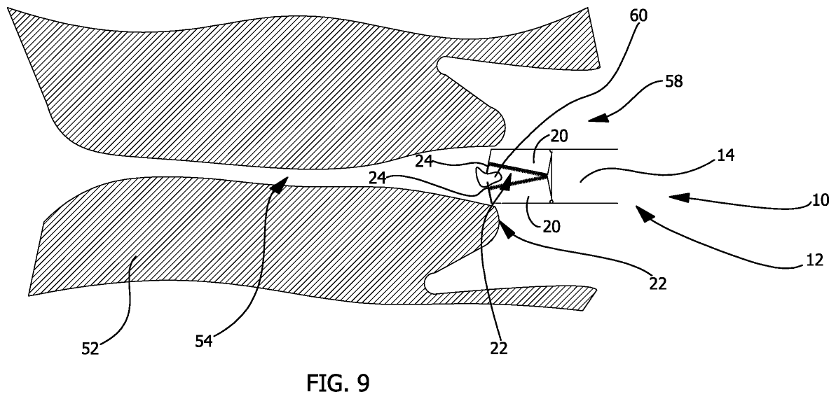

As illustrated in FIGS. 8 and 9, another embodiment of the disclosure comprises a method of retrieving a biopsy tissue 60 from a cervix 52 from an endocervical canal 54 of the cervix 52. The method includes inserting a forceps 12 and a dilator 34 into a vaginal canal 58, wherein the forceps 12 houses the dilator 34. The dilator 34 is caused to extend out of the forceps 12. An insertion member 40 of the dilator 34 is inserted into the endocervical canal 54 through a cervical os 56. The insertion member 40 has a conical shape, and a tip 42 of the insertion member 40 leads a base 44 of the insertion member 40 when the insertion member 40 is inserted into the endocervical canal 54. A conical surface 45 of the insertion member 40 gradually dilates the cervical os 56 as the insertion member 40 is inserted farther into the endocervical canal 54. The base 44 of the insertion member 40 has a width of less than 0.30 inches and a length of the insertion member 40 from the tip 42 to the base 44 is at most 0.60 inches. The insertion member 40 is inserted into the endocervical canal 54 to dilate the cervical os 56 to a diameter of less than 0.30 inches.

The dilator 34 is retracted into the forceps 12, and a pair of jaws 20 of the forceps 12 is inserted into the endocervical canal 54 through the cervical os 56. The jaws 20 are closed against the biopsy tissue 60 to sever the biopsy tissue 60 from the cervix 52 and hold the biopsy tissue 60 with the pair of jaws 20. The forceps 12 and the dilator 34 are extracted from the vaginal canal 58.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A dilation and biopsy apparatus comprising:
a forceps comprising:
a shaft; and
a pair of jaws being pivotally coupled to a front end of the shaft, the pair of jaws being pivotable to alternately open and close; and
a dilator being mounted to the shaft of the forceps, the dilator comprising:
a rod being positioned in the shaft and being linearly movable alternately forwardly and rearwardly with respect to the shaft; and
an insertion member being coupled to a forward end of the rod, the insertion member being movable alternately into and out of the shaft through the front end of the shaft and between the pair of jaws.

2. The apparatus of claim 1, wherein the insertion member having a conical shape, a tip of the conical shape pointing forwardly with respect to the shaft.

3. The apparatus of claim 2, wherein a base of the insertion member has a width of at most 0.30 inches.

4. The apparatus of claim 3, wherein the insertion member has a length from the tip to the base of at most 0.60 inches.

5. The apparatus of claim 1, wherein the dilator further comprises a rod actuator being positioned in the shaft and being movable to move the rod.

6. The apparatus of claim 5, wherein the rod actuator has internal threads which engage external threads of the rod whereby the rod actuator is rotatable to cause the rod to move linearly with respect to the shaft.

7. The apparatus of claim 8, wherein the dilator further comprises a knob which is coupled to the rod actuator and is positioned rearwardly of the shaft.

8. The apparatus of claim 1, wherein the pair of jaws defines a space therebetween having a size such that the pair of jaws are configured to hold a biopsy tissue in the space.

9. The apparatus of claim 8, wherein the pair of jaws has interlocking teeth positioned distally from the shaft, the interlocking teeth having cutting edges such that the interlocking teeth are configured for severing the biopsy tissue from a target lumen when the pair of jaws close onto the biopsy tissue.

10. The apparatus of claim 1, wherein the forceps further comprises a trigger being coupled to a rear end of the shaft, the trigger being operatively coupled to the pair of jaws to alternately open and close the jaws.

11. The apparatus of claim 10, wherein the forceps further comprises a pair of actuator arms being slidably mounted in the shaft, each actuator arm of the pair of actuator arms being coupled to the trigger and an associated jaw of the pair of jaws, the trigger being movable to cause the pair of actuator arms to move linearly through the shaft and urge the pair of jaws to alternately open and close.

12. The apparatus of claim 11, wherein the forceps further comprises a handle being coupled to the rear end of the shaft, the trigger being pivotable alternately toward and away from the handle.

13. The apparatus of claim 1, wherein the forceps further comprises a bias member being mounted to cause the pair of jaws to close.

14. The apparatus of claim 12, wherein the forceps further comprises a bias member being coupled to the trigger and the handle to bias the trigger away from the handle, thereby causing the pair of jaws to close.

15. A dilation and biopsy apparatus comprising:
a forceps comprising:
a shaft;
a pair of jaws being pivotally coupled to a front end of the shaft, the pair of jaws being pivotable to alternately open and close, the pair of jaws defining a space therebetween having a size such that the pair of jaws are configured to hold a biopsy tissue in the space, the pair of jaws having interlocking teeth positioned distally from the shaft, the interlocking teeth having cutting edges such that the interlocking teeth are configured for severing the biopsy tissue from a target lumen when the pair of jaws close onto the biopsy tissue;
a trigger being coupled to a rear end of the shaft, the trigger being operatively coupled to the pair of jaws to alternately open and close the jaws, the trigger being pivotable with respect to the shaft;
a pair of actuator arms being slidably mounted in the shaft, each actuator arm of the pair of actuator arms being coupled to the trigger and an associated jaw of the pair of jaws, the trigger being pivotable to cause the pair of actuator arms to move linearly through the shaft and urge the pair of jaws to alternately open and close;
a handle being coupled to the rear end of the shaft, the trigger being pivotable alternately toward and away from the handle; and
a bias member being coupled to the trigger and the handle to bias the trigger away from the handle, thereby causing the pair of jaws to close; and
a dilator being mounted to the shaft of the forceps, the dilator comprising:
a rod being positioned in the shaft and being linearly movable alternately forwardly and rearwardly with respect to the shaft;
an insertion member being coupled to a forward end of the rod, the insertion member having a conical shape, a tip of the conical shape pointing forwardly with respect to the shaft, the insertion member being movable alternately into and out of the shaft through the front end of the shaft and between the pair of jaws, a base of the insertion member having a width of at most 0.30 inches, the insertion member having a length from the tip to the base of at most 0.60 inches;
a rod actuator being positioned in the shaft and being movable to move the rod, the rod actuator having internal threads which engage external threads of the rod whereby the rod actuator is rotatable to cause the rod to move linearly with respect to the shaft; and
a knob being coupled to the rod actuator and being positioned rearwardly of the shaft.

16. A method of retrieving a biopsy tissue from a cervix from an endocervical canal of the cervix, the method comprising:
inserting a forceps and a dilator into a vaginal canal, wherein the forceps houses the dilator;
extending the dilator out of the forceps;
dilating a cervical os of the endocervical canal by inserting an insertion member of the dilator into the endocervical canal through the cervical os;
retracting the dilator into the forceps;
inserting a pair of jaws of the forceps into the endocervical canal through the cervical os;
closing the pair of jaws against the biopsy tissue to sever the biopsy tissue from the cervix and hold the biopsy tissue with the pair of jaws; and
extracting the forceps and the dilator from the vaginal canal.

17. The method of claim 16, wherein the insertion member has a conical shape, wherein a tip of the insertion member leads a base of the insertion member as the insertion member is inserted into the endocervical canal, whereby a conical surface of the insertion member gradually dilates the cervical os as the insertion member is inserted farther into the endocervical canal.

18. The method of claim 16, wherein the step of dilating the cervical os further comprises dilating the cervical os to a diameter of less than 0.30 inches.

19. The method of claim 17, wherein the base of the insertion member has a width of less than 0.30 inches.

20. The method of claim 19, wherein the insertion member has a length from the tip to the base of at most 0.60 inches.
